(19) United States
(12) Patent Application Publication
(10) Pub. No.: US 2005/0203331 A1
(43) Pub. Date: Sep. 15, 2005

(54) Neurosurgical instrument with gamma ray detector

(76) Inventors: Matthew P. Szapucki, Toms River, NJ (US); Nancy E. Rosenblum, Colts Neck, NJ (US); Bruce R. Rosenblum, Colts Neck, NJ (US)

Correspondence Address: Kenneth Watov;WATOV & KIPNES, P.C., P.O. Box 247, Princeton Junction, NJ, 08550, US

(21) Appl. No.: 11/075,066

(22) Filed: Mar. 7, 2005

Related U.S. Application Data
(60) Provisional application No. 60/551,582, filed on Mar. 9, 2004.

(51) Int. Cl. A61M 36/14; A61K 51/00

(52) U.S. Cl. 600/1

(57) ABSTRACT

A neurosurgical instrument includes a radiation detecting sensor adjustably positioned proximate a distal end of an arm of the instrument. A controller is programmed to receive and process electrical signals from the sensor. The controller operates to control an audio tone generator to emit a tone of higher intensity as the surgeon moves the sensor and its associated coagulator closer to radioactively tagged malignant tissue or a brain tumor of a patient, and of lower intensity as the coagulator moves away from the tissue or tumor, thereby permitting the surgeon to accurately locate and remove the malignant tissue or brain tumor. Also, in place of or in combination with the audio tone generator, a light emitter provides for emitting light of intensity directly related to the distance the sensor is from the malignant tissue or brain tumor tagged with a radioactive isotope.

### RELATED APPLICATION

The present invention is related to and takes priority from U.S. Provisional Patent Application Ser. No. 60/551,582, filed on Mar. 9, 2004, for “COAGULATOR WITH GAMMA RAY DETECTOR,” the teachings of which are incorporated herein by reference to the extent that they do not conflict herewith.

### FIELD OF THE INVENTION

The present invention relates generally to neurological instruments, and more particularly to such instruments that include mechanisms for permitting a surgeon to more readily locate and remove malignant brain tissue and/or brain tumors from a patient.

### BACKGROUND OF THE INVENTION

It is important for surgeons to have the ability to remove the maximum amount, and preferably all of a malignant tumor or cancerous tissue. It is known in the art to inject patients with a radioactive isotope prior to cancer surgery, whereafter a radiation detection device can be utilized by the surgeon in order to locate cancerous tissue or tumors where the isotope tends to accumulate. Use of radioactive isotopes, such as thallium, along with radiation detectors has successfully been used in surgically removing cancerous lymph nodes from various areas of a patient's body. However, because of the restricted space available to a surgeon for operating on malignant brain tissue or tumors, the present apparatus available for radioactive isotope assisted surgery is not readily useable in neurosurgery. Yet, in order to improve a patient's chances of survival, and reducing the recurrence of a cancerous brain tumor, it is particularly important that a neurosurgeon have the ability to preferably completely remove the brain tumor, and to at least have the ability to locate and remove major portions of malignant brain tissue.

### SUMMARY OF THE INVENTION

In one embodiment of the present invention, a standard neurosurgical instrument, such as a coagulator, forceps, or cavitational ultrasonic surgical aspirator, for example, is modified to incorporate therewith a gamma ray detector, or other useable radiation detector. During surgery, a patient is injected with a known radioactive isotope that tends to accumulate in malignant brain tissue or tumors, for permitting a neurosurgeon to expose the affected area of the brain, and thereafter position the instrument, having a radiation sensor attached to and in close proximity to an extended arm thereof, for example, for permitting the surgeon to quickly locate the radioactive isotope infiltrated tissue or tumor for removal.

In another embodiment of the invention, a radiation sensor is mounted proximate the distal end of an arm of an associated neurosurgical instrument, such as a coagulator electrode arm, for example. The other end of the arm is secured to a lower portion/of the coagulator main frame. Note that for purposes of example, the invention is described in association with a coagulator, but is not meant to be limited thereto. This arrangement permits a surgeon to position the radiation detector tip close to one of the electrodes of the coagulator. An electrical cable has one end run through the associated arm and electrically connected to the radiation sensor, a portion of the cable being attached to the associated coagulator arm. The other end of the cable is electrically connected to electronic processing equipment, a controller, for receiving signals from the radiation sensor, and processing the same. When the radiation sensor receives radiation signals above a predetermined level, the controller will trigger a sound and/or light emitting device to indicate to the surgeon that the coagulator is in the vicinity of a brain tumor and/or malignant brain tissue that has absorbed a radioactive isotope. In another embodiment, the controller adjusts the intensity of the audible alarm or light indicator to increase as the sensor approaches radioactively tagged tissue, and decrease as the sensor moves away from the tissue.

### BRIEF DESCRIPTION OF THE DRAWINGS

Various embodiments of the present invention are described below in detail with reference to the drawings, in which like items are identified by the same reference designation, wherein:

FIG. 1 is a pictorial view of a standard type of coagulator used in neurosurgery to which a radiation sensor has been added for one embodiment of the invention;

FIG. 2 shows an embodiment of the invention for including a radiation sensor on a neurosurgical instrument, such as a coagulator, in a substantially fixed manner for permitting sterilization of the assembly after each use;

FIG. 3 is a pictorial view of a mechanical attachment device for use in the embodiment of the invention of FIG. 2, whereby the attachment device itself represents another embodiment of the invention;

FIG. 4 is a cross-sectional view taken along 4-4 of FIG. 3 with an associated cap screw shown in an exploded assembly view configuration;

FIG. 5 shows another embodiment of the invention for removably attaching a radiation detector to a neurosurgical instrument, such as a coagulator, for providing an easily disposable configuration for the radiation sensor;

FIG. 6 shows a cross-sectional view taken along 6-6 of FIG. 5, modified for purposes of illustration to be an exploded assembly view configuration; and

FIG. 7 shows a pictorial view of the clip configuration of FIGS. 5 and 6, for the disposable embodiment of the invention.

### DETAILED DESCRIPTION OF THE INVENTION

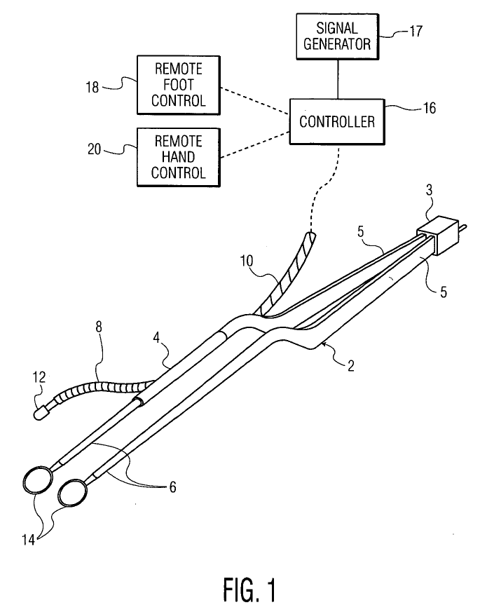

With reference to FIG. 1, a standard coagulator 2 for use in neurological surgery is shown modified for one embodiment of the invention. As previously mentioned, the various embodiments of the invention, for ease of illustration, are described in association with a neurosurgical coagulator 2, but the invention is not meant to be so limited, in that it can be applied for use with other neurosurgical instruments. Note the coagulator 2 includes two arm extensions 5 terminated to a plug 3, to permit the coagulator to be disconnected from its power source and controller (not shown) for purposes of sterilization after use. Coagulator 2 also includes two arms 6 formed as part of and protruding from respective arm extensions 5, as shown. Individual electrodes are connected to the free ends of each protruding arm 6. The modifications include, in this example, using an electrically insulated sheath 4 to hold tightly against a lower portion of one arm 6 of the coagulator 2, a lower portion of a tubular flexible arm 8, and a portion of an electrical cable 10, as shown. One end of the cable 10 is threaded through the center of the flexible arm 8 for electrical connection to the radiation sensor 12. The other end of the cable 10 is connected to a controller 16. In this example, the radiation sensor 12 is a gamma ray sensor. However, depending upon the type of radioactive isotope used in the surgical procedure, the radiation sensor 12 can be other than a gamma ray sensor, for example, a beta ray sensor. Accordingly, the various embodiments of the invention are not meant to be limited to only a gamma ray sensor 12, or sensing gamma rays, or neurosurgical coagulators.

Certain radiation sensors are capable of sensing both beta and gamma rays to given degrees of sensitivity. The insulated sheath 4 can be provided by shrink wrap tubing, for example, or other suitable dielectric material, in one embodiment of the invention. Note also that in another embodiment of the invention the radiation sensor can be incorporated into one arm 6 of a coagulator, with the radiation detecting tip or element 12 being electrically and thermally insulated from the associated electrode operating element 14 of the coagulator 2.

The electrical cable 10 has its other end electrically connected to an electronic control device or controller 16 that processes signals from the sensor 12 via an included programmable microprocessor (not shown), for example, and also includes an output signal generator 17 for providing a surgeon with either an acoustical alarm and/or light emitter of varying intensity depending upon whether the radiation sensor 12 is being moved closer to (increasing intensity) or further from (decreasing intensity) malignant brain tissue or a brain tumor, in this example, that has absorbed the aforesaid radioactive isotope. The cable 10 may include coaxial cable to conduct signals from sensor 12 to the controller 16, and shield such signals from electrical interference. Note also that the signal generator 17 can be provided as either a separate module, or in a module also including controller 16.

In another embodiment of the invention, the radiation sensor 12 is no larger than one-quarter inch in diameter for insuring its easy affixation to a desired neurological instrument such as a coagulator 2, as shown. It is expected that the electronic controller 16, in a preferred embodiment, will incorporate wireless features, such as battery backup, low battery alert, adjustment of the sensitivity of the device, and adjustment of the acoustical sound alerting portion of the system. Note that presently available coagulators typically include foot pedal controllers for operating the coagulator device. Also, the present controller 16 may be completely self powered, and attached to the surgeon's operating uniform or garb.

In another embodiment of the invention, the controller 16 and signal generator 17 can be contained in a single module for belt mounting to the surgeon in a sterile manner. Also, wireless foot or hand controllers 18, 20, respectively, may be included for permitting the surgeon to control the main controller 16 of the radiation detector without the encumbrance of control cables around the surgical table. To avoid false readings, the controller 16 can be selectively turned on or off to avoid false readings.

It should be noted that presently available coagulator apparatus provide for both a cutting mode for removing tissue, and a coagulation mode for closing off blood vessels at the surgical site. An example of presently available neurosurgical coagulators is the “IsoCool” line of Codman, Raynham, Mass. 02767, which includes both bipolar forceps, and electrical bipolar generators, and controllers. As previously indicated, the present radiation detection system can be added to such standard coagulator devices or apparatus 2, or can be incorporated into a newly designed coagulator device, or incorporated into other neurosurgical instruments.

In one embodiment of the invention, the radiation sensor 12 is likely to be provided by a 716 Gamma Detector of LND, Inc., Oceanside, N.Y. Also, the flexible arm 8 is likely to be provided by a “flexible arm” of Uniprize International, Terryville, Conn.

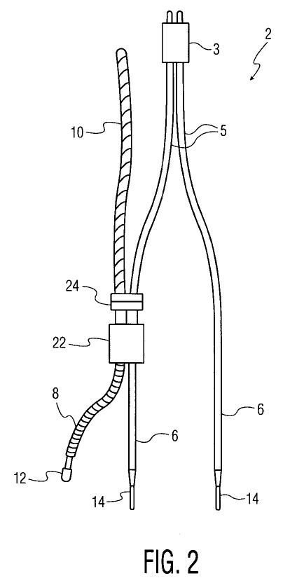

In another embodiment of the invention, as shown in FIG. 2, a radiation sensor 12 having its electrical cable 10 enclosed within a flexible arm 8, is semipermanently attached to an arm of a neurosurgical instrument, such as arm 6 of a coagulator, for example. The mechanical attachment is provided by an attachment device 22, which will be described in greater detail below. Also, the electrical cable 10 of the radiation sensor 12 is moveably attached via the use of a known plug-in connector 24, suitable for such use. In this embodiment of the invention, the cable 10 can be unplugged via the connector 2, and the assembly of the radiation sensor 12, with flexible arm 8, and coagulator's arms 6, can be sterilized for reuse.

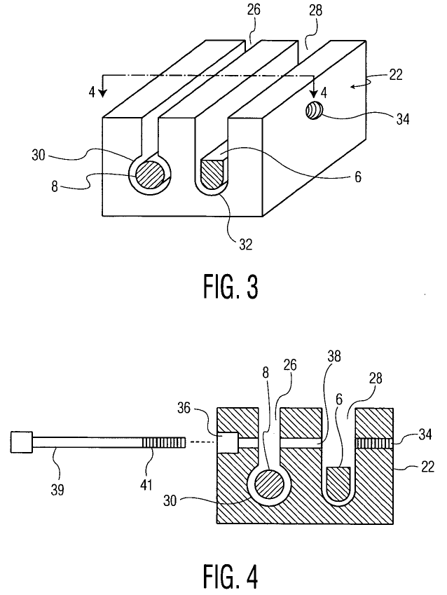

FIG. 3 shows a pictorial view of the mechanical attachment device 22. The device 22 can be made from a number of materials, including but not limited to aluminum, stainless steel, and appropriate plastic materials. As shown, the attachment device 22 includes a pair of spaced apart open slotways 26, 28, respectively. The bottom of slotway 26 terminates into a cylindrical cavity 30 dimensioned for snugly carrying therein the flexible arm 8. The open slotway 28 is configured for snugly receiving and retaining a portion of an arm of an associated neurological instrument, such as coagulator arm 6, in this example, as shown. A cross section of the attachment device 22 taken along 4-4 is shown in FIG. 4. As shown, a cap screw 39 is inserted through the successive holes 36, 38, and into threaded hole 34, for permitting the cap screw 39 to have its threaded end 41 screwed into threaded hole 34 for causing the circular cavity 30, and slotways 26 and 28 to be dimensionally reduced for squeezing or compressing against the portion of the flexible arm 8, and the portion of the instrument arm 6, for rigidly retaining the two within the attachment device 22. In this manner, the flexible sheet or arm 8 and the arm 6 of a coagulator, in this example, are rigidly secured together. After use of the coagulator 2 in combination with the radiation sensor 12 and tubular flexible arm 8, the cable 10 is disconnected from the assembly via connector 24, and the opposing coagulator arms 6, in this example, are unplugged from their main coagulator body 2, for permitting sterilization of the detached elements for later reuse.

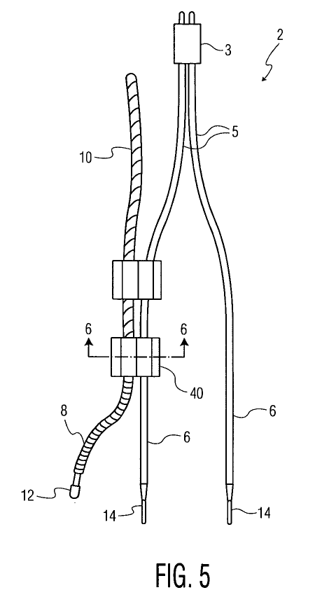

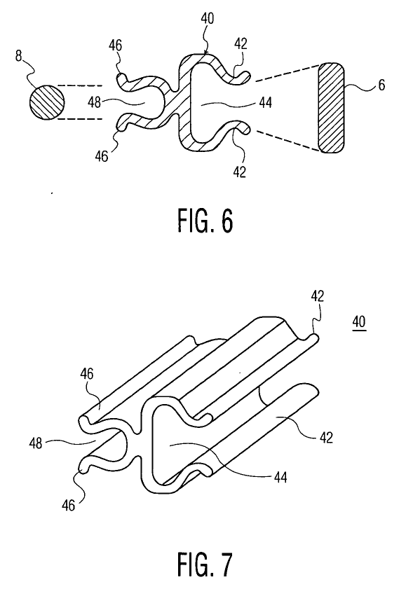

In another embodiment of the invention, the radiation sensor 12 and associated tubular flexible arm 8, and cable 10 are removably attached to a neurosurgical instrument such as to a coagulator arm 6, in this example, as shown in FIG. 5. In this embodiment, two clips 40 are used for removably attaching the sensor assembly 8, 10, 12 to the coagulator arm 6. In FIG. 6, a cross section of a clip 40 taken along 6-6 of FIG. 5 is shown in an exploded assembly view configuration with a tubular flexible arm 8 and coagulator arm 6 portion. The clip 40 includes opposing flexible fingers 42 for permitting a portion of a coagulator arm 6 or other neurosurgical instrument portion to be snapped into the receiving area 44 thereof, for securely retaining the coagulator arm 6 portion, in this example. The other end of the clip 40 includes a pair of opposing flexible fingers 46 for permitting a portion of the tubular flexible arm or shaft 8 to be snapped into a receiving area 48 of a clip 40, and securely held therein. FIG. 7 shows a pictorial view of the clip 40. It is expected that typically two or more such clips 40 will be utilized for securing the radiation sensor assembly 8, 10, 12 to a portion of a neurosurgical instrument, such as coagulator 2. Note that the clip 40 can be fabricated from any suitable material, including polycarbonate, nylon, and so forth.

Prior to using a neurosurgical instrument fitted with the radiation sensor assembly 8, 10, 12 of the present invention, a patient being prepared for brain surgery to remove a malignant or cancerous tumor, must first typically have the patient injected with an appropriate radioactive tracer that after injection will travel to and accumulate in the malignant tumor. Thallium 201 has been used for a number of years in nuclear medicine. Thallium is known to emit gamma rays. Accordingly, when this radioactive isotope is utilized, the radiation sensor 12 must be capable of detecting gamma rays. The method of administration of Thallium 201, and other radioactive isotopes, is well known by anesthesiologists. Accordingly, for the sake of brevity, a description of a typical procedure for targeting brain tumors with a radioactive tracer, such as Thallium 201, will not be described herein. After the radioactive tracer has had sufficient time to accumulate in the brain tumor, the surgeon can then begin use of the present invention in association with a neurosurgical instrument for assisting in the removal of the malignant tissue. Thallium 201 is produced by Amersham located in Livingston, N.J.

Although various embodiments of the invention have been shown and described above, they are not meant to be limiting. As previously indicated, for purposes of illustration various embodiments of the invention have been described in association with a neurosurgical coagulator, whereas the invention can also be practiced in association with other neurosurgical instruments, such as forceps, ultrasonic surgical aspirators, and rongeurs, for example. Those of skill in the art may recognize certain modifications to these embodiments, which modifications are meant to be covered by the spirit and scope of the appended claims.

1. A neurosurgical apparatus for use in the removal of malignant brain tissue and/or brain tumors, said apparatus comprising:
a handheld neurosurgical instrument including at least a first arm having a distal end for positioning in contact with or in close proximity to malignant brain tumors and/or tissue, the distal end being configured for providing a desired neurosurgical function; and
a radiation detection system including a radiation sensor mounted upon said first arm proximate the distal end thereof, said system being operable to produce either one or a combination of an audio signal or visual signal of increasing intensity as said radiation sensor is moved closer to malignant brain tissue and/or tumors that have previously absorbed or been infiltrated with a radioactive isotope injected into a patient prior to surgery.

2. The neurosurgical apparatus of claim 1, wherein said radiation detection system further includes:
a first electrical cable having one end electrically connected to said radiation sensor;
a tubular flexible arm containing a portion of said electrical cable, said radiation sensor extending from a distal end of said tubular flexible arm, said first cable having a portion extending from a proximal end of said flexible arm; and
attachment means for rigidly attaching a portion of said tubular flexible arm proximate its proximal end to said first arm of said neurological instrument, whereby said flexible arm permits adjustment within a range of the positioning of said radiation sensor relative to the distal end of said first arm of said neurosurgical instrument.

3. The neurosurgical apparatus of claim 2, wherein said attachment means consist of shrink wrap tubing.

4. The neurosurgical apparatus of claim 2, wherein said attachment means include:
a block of semirigid material including:
a first channel extending between opposing side portions of said block, open from a top portion thereof toward a bottom portion thereof, said first channel being configured to snugly receive a portion of said tubular flexible arm;
a second channel adjacent and parallel to said first channel, said second channel extending into a top portion of said block toward the bottom portion thereof, between said opposing side portions thereof, said second channel being configured to snugly receive a portion of said first arm of said neurosurgical instrument; and
means for simultaneously and controllably reducing the widths of said first and second channels for rigidly securing together said flexible arm and said first arm of said neurosurgical instrument.

5. The neurosurgical apparatus of claim 4, further including:
a connector for removably mechanically and electrically attaching a second electrical cable to the portion of said first electrical cable extending from the proximal end of said flexible arm, thereby facilitating sterilization of said neurosurgical instrument with said radiation sensor, tubular flexible arm, and first electrical cable.

6. The neurosurgical apparatus of claim 2, wherein said attachment means includes: at least one clip consisting of semiflexible material having memory, said clip including:
a pair of opposing first fingers for snapping onto and rigidly retaining a portion of said tubular flexible arm; and
a pair of opposing second fingers co-joined to said pair of opposing first fingers, said second fingers being configured for snapping onto and rigidly retaining a portion of said first arm of said neurosurgical instrument.

7. The neurosurgical apparatus of claim 1, wherein said radiation detection system further includes:
controller means electrically connected to said radiation sensor for receiving and processing signals therefrom; and
audio and/or visual signal generating means operated by said controller means for producing said audio signal.

8. The neurosurgical apparatus of claim 1, wherein said handheld neurosurgical instrument is a coagulator.

9. The neurosurgical apparatus of claim 1, wherein said radiation detection system further includes a wireless foot operated remote control device.

10. The neurosurgical apparatus of claim 1, wherein said radiation detection system further includes a wireless hand operated remote control device.

11. The neurosurgical apparatus of claim 1, wherein said radiation detecting sensor is a gamma ray detecting sensor.

12. Radiation sensing apparatus for attachment to an arm of a neurosurgical instrument employed in the removal of a brain tumor or malignant tissue of a patient, said patient prior to surgery being injected with a radioactive isotope for tagging said brain tumor and/or malignant tissue, said apparatus comprising:
a radiation sensor mounted upon and proximate to a distal end of said arm of said neurosurgical instrument;
controller means for receiving and processing signals from said radiation sensor; and
audio and/or visual signal generating means operable by said controller means for alerting a surgeon that the radiation sensor and distal end of said arm of said neurosurgical instrument are being moved closer to, or away from, or have arrived in close proximity to the malignant tissue and/or tumor.

13. The radiation sensing apparatus of claim 12, further including:
a first electrical cable having one end electrically connected to said radiation sensor;
a tubular flexible arm containing a portion of said electrical cable, said radiation sensor extending from a distal end of said flexible arm, said first cable having a portion extending from a proximal end of said flexible arm; and
attachment means for rigidly attaching a portion of said flexible arm proximate its proximal end to said arm of said neurological instrument, whereby said flexible arm permits adjustment within a range of the positioning of said radiation sensor relative to the distal end of said arm of said neurosurgical instrument.

14. The radiation sensing apparatus of claim 13, wherein said attachment means consists of shrink wrap tubing.

15. The radiation sensing apparatus of claim 13, wherein said attachment means includes: a block of semirigid material including:
a first channel extending between opposing side portions of said block, open from a top portion thereof toward a bottom portion thereof, said first channel being configured to snugly receive a portion of said flexible arm;
a second channel adjacent and parallel to said first channel, said second channel extending into a top portion of said block toward the bottom portion thereof, between said opposing side portions thereof, said second channel being configured to snugly receive a portion of said arm of said neurosurgical instrument; and
means for simultaneously and controllably reducing the widths of said first and second channels for rigidly securing together said flexible arm and said arm of said neurosurgical instrument.

16. The radiation sensing apparatus of claim 15, further including:
a connector for removably mechanically and electrically attaching a second electrical cable to the portion of said first electrical cable extending from the proximal end of said flexible arm, thereby facilitating sterilization of said neurosurgical instrument with said radiation sensor, tubular flexible arm, and first electrical cable.

17. The radiation sensing apparatus of claim 13, wherein said attachment means includes: at least one clip consisting of semiflexible material having memory, said clip including:
a pair of opposing first fingers for snapping onto and rigidly retaining a portion of said tubular flexible arm; and
a pair of opposing second fingers co-joined to said pair of opposing first fingers, said second fingers being configured for snapping onto and rigidly retaining a portion of said arm of said neurosurgical instrument.

18. The radiation sensing apparatus of claim 12, wherein said handheld neurosurgical instrument is a coagulator.

19. The radiation sensing apparatus of claim 12, wherein said radiation sensor consists of a gamma ray detector.

20. A neurosurgical coagulator system for permitting a surgeon to accurately locate and remove a brain tumor and/or malignant brain tissue from a patient, comprises:
first and second arms, each having first and second ends;
first and second electrodes mounted upon the first ends of said first and second arms, the second ends of said first and second arms being secured to a common substrate in a manner to retain said electrodes spaced apart and in opposition to one another, whereby the first and second arms are flexible;
radiation detection means including:
a radiation sensor mounted on said coagulator in close proximity to one of said first and second electrodes;
controller means for receiving and processing signals from said radiation sensor; and
audio and/or visual signal generating means operated by said controller means for alerting a surgeon that said first and second electrodes are being moved closer to, or away from, or have arrived in close proximity to malignant tissue that has absorbed or been infiltrated with a portion of a radioactive isotope previously injected into a patient.

21. The surgical coagulator system of claim 20, wherein the radioactive isotope is thallium.

22. The surgical coagulator system of claim 21, wherein said radiation detecting sensor is a gamma ray detecting sensor.

23. A method for permitting a surgeon to accurately locate and remove malignant tissue from a patient, comprising the steps of:
adjustably mounting a radiation sensor in close proximity to a distal end of an arm of a handheld neurosurgical instrument;
injecting a radioactive isotope into a patient, which isotope is known to lodge in the brain tumor and/or malignant brain tissue of interest; and
processing electrical signals from said radiation sensor to provide either one or a combination of an audible and/or visual signal to a surgeon indicative of the radiation sensor being moved toward, away from, or positioned proximate the tumor and/or malignant tissue.

24. The method of claim 23, wherein said radioactive isotope is Thallium.

25. The method of claim 23, wherein said radiation sensor is a gamma ray sensor.
